(19) United States
(12) Patent Application Publication
(10) Pub. No.: US 2026/0241136 A1
(43) Pub. Date: Aug. 20, 2026

(54) MOOD STATE

(71) Applicant: Nicoventures Trading Limited, London (GB)

(72) Inventors: Jeffrey Sean SMITH, London (GB); Sean LUKAN, London (GB); Laura SMITH, London (GB); Chloe CORDERY, London (GB)

(21) Appl. No.: 19/164,758

(22) PCT Filed: Mar. 15, 2024
(86) PCT No.: PCT/GB2024/050695

§ 371 Date: Sep. 12, 2025

Related U.S. Application Data
(63) Continuation of application No. 18/184,142, filed on Mar. 15, 2023, now abandoned.

(51) Int. Cl. A61M 21/00

(52) U.S. Cl. CPC A61M 21/00; A61M 2021/0077; A61M 2205/3303; A61M 2205/3553; A61M 2209/088

(57) ABSTRACT

A method, apparatus and computer program is described comprising: one or more sensors for obtaining real-time physiological data for use in determining a mood state of a user; communicating means for providing said sensor data to a remote data processing module and for receiving input from said remote data processing module, wherein the remote data processing module comprises a model for determining a future mood state of the user based on said sensor data; and a delivery mechanism for delivering one or more active compounds to influence, manage or control the mood state of said user, wherein delivery of said compounds is controlled based on the input received from said remote data processing module.

### FIELD

The present specification relates to management and control of a mood state of user.

### BACKGROUND

Many systems are known for delivering one or more active compounds to a user that seek to change or control a mood state of the user. There remains a need for further developments in this field.

### SUMMARY

In a first aspect, this specification describes a system comprising: one or more sensors for obtaining real-time physiological data for use in determining a mood state of a user; communicating means for providing said sensor data to a remote data processing module and for receiving input from said remote data processing module, wherein the remote data processing module comprises a model for determining a future mood state of the user based on said sensor data; and a delivery mechanism for delivering one or more active compounds to influence, manage or control the mood state of said user, wherein delivery of said compounds is controlled based on the input received from said remote data processing module.

The system may further comprise a control module for determining said active compound delivery based, at least in part, on the input received from said remote data processing module.

The said remote data processing module may comprise a control module for determining said active compound delivery of said active compounds.

In the use of the system, the user may be a virtual world (e.g. the Metaverse).

In some example embodiments, the said sensors are mounted within a user headset (such as a virtual reality, augmented reality, mixed reality, extended reality or similar headset).

The said sensors may comprise one or more of: one or more transparent optical imaging cameras; one or more thermal sensors; and one or more non-thermal sensors.

The remote data processing module may comprise an artificial intelligence or machine learning model.

The delivery module may comprise an aerosol delivery mechanism.

In a second aspect, this specification describes a method comprising: obtaining real-time physiological data for use in determining a mood state of a user; providing said sensor data to a remote data processing module, wherein the remote data processing module comprises a model for determining a future mood state of the user based on said sensor data; receiving input from said remote data processing module; and delivering one or more active compounds to influence, manage or control the mood state of said user, wherein delivery of said compounds is controlled based on the input received from said remote data processing module. In the use of the method, the user may be in a virtual world (e.g. the Metaverse).

The method may further comprise determining said active compound delivery based, at least in part, on the input received from said remote data processing module.

The remote data processing module may comprise an artificial intelligence or machine learning model.

The delivery module may comprises an aerosol delivery mechanism.

In a third aspect, this specification describes computer-readable instructions which, when executed by a computing apparatus, cause the computing apparatus to perform (at least) any method as described herein (including the method of the second aspect described above).

In a fourth aspect, this specification describes a computer-readable medium (such as a non-transitory computer-readable medium) comprising program instructions stored thereon for performing (at least) any method as described herein (including the method of the second aspect described above).

In a fifth aspect, this specification describes an apparatus comprising: at least one processor; and at least one memory including computer program code which, when executed by the at least one processor, causes the apparatus to perform (at least) any method as described herein (including the method of the second aspect described above).

In a sixth aspect, this specification describes a computer program comprising instructions for causing an apparatus to perform at least the following: obtaining real-time physiological data for use in determining a mood state of a user; providing said sensor data to a remote data processing module, wherein the remote data processing module comprises a model for determining a future mood state of the user based on said sensor data; receiving input from said remote data processing module; and delivering one or more active compounds to influence, manage or control the mood state of said user, wherein delivery of said compounds is controlled based on the input received from said remote data processing module.

### BRIEF DESCRIPTION OF THE DRAWINGS

Example embodiments will now be described, by way of example only, with reference to the following schematic drawings, in which:

FIG. 1 is a block diagram of a system in accordance with an example embodiment;

FIG. 2 is a flow chart showing an algorithm in accordance with an example embodiment;

FIG. 3 is a block diagram demonstrating example types of immersion in accordance with example embodiments;

FIG. 4 shows a user wearing a headset and inhaler in accordance with an example embodiment;

FIG. 5 is a block diagram of a system in accordance with an example embodiment;

FIG. 6 is a flow chart showing an algorithm in accordance with an example embodiment;

FIG. 7 is a block diagram of an aerosol generating device in accordance with an example embodiment;

FIG. 8 is a block diagram of a system in accordance with an example embodiment;

FIG. 9 shows a user in accordance with an example embodiment;

FIG. 10 shows a sensor used in some example embodiments;

FIG. 11 shows a user wearing a headset in accordance with an example embodiment;

FIG. 12 is a block diagram of a system in accordance with an example embodiment;

FIG. 13 is a flow chart showing an algorithm in accordance with an example embodiment;

FIG. 14 is a block diagram of a system in accordance with an example embodiment;

FIG. 15 is a block diagram of a neural network used in some example embodiments;

FIG. 16 is a block diagram showing an aerosol delivery mechanism in accordance with an example embodiment; and

FIG. 17 is a block diagram of a processing system that may be used to implement one or more of the example embodiments.

### DETAILED DESCRIPTION

As used herein, the term “delivery mechanism” is intended to encompass systems that deliver a substance to a user, and includes:
non-combustible aerosol provision systems that release compounds from an aerosolisable material without combusting the aerosolisable material, such as electronic cigarettes, tobacco heating products, and hybrid systems to generate aerosol using a combination of aerosolisable materials; and
articles comprising aerosolisable material and configured to be used in one of these non-combustible aerosol provision systems.

According to the present disclosure, a “non-combustible” aerosol provision system is one where a constituent aerosol-generating material of the aerosol provision system (or component thereof) is not combusted or burned in order to facilitate delivery of at least one substance to a user.

In some embodiments, the delivery system is a non-combustible aerosol provision system, such as a powered non-combustible aerosol provision system.

In some embodiments, the non-combustible aerosol provision system is an electronic cigarette, also known as a vaping device or electronic nicotine delivery system (END), although it is noted that the presence of nicotine in the aerosol-generating material is not a requirement.

In some embodiments, the non-combustible aerosol provision system is an aerosol-generating material heating system, also known as a heat-not-burn system. An example of such a system is a tobacco heating system.

In some embodiments, the non-combustible aerosol provision system is a hybrid system to generate aerosol using a combination of aerosol-generating materials, one or a plurality of which may be heated. Each of the aerosol-generating materials may be, for example, in the form of a solid, liquid or gel and may or may not contain nicotine. In some embodiments, the hybrid system comprises a liquid or gel aerosol-generating material and a solid aerosol-generating material. The solid aerosol-generating material may comprise, for example, tobacco or a non-tobacco product.

Typically, the non-combustible aerosol provision system may comprise a non-combustible aerosol provision device and a consumable for use with the non-combustible aerosol provision device.

In some embodiments, the disclosure relates to consumables comprising aerosol-generating material and configured to be used with non-combustible aerosol provision devices. These consumables are sometimes referred to as articles throughout the disclosure.

In some embodiments, the non-combustible aerosol provision system, such as a non-combustible aerosol provision device thereof, may comprise a power source and a controller. The power source may, for example, be an electric power source or an exothermic power source. In some embodiments, the exothermic power source comprises a carbon substrate which may be energised so as to distribute power in the form of heat to an aerosol-generating material or to a heat transfer material in proximity to the exothermic power source.

In some embodiments, the non-combustible aerosol provision system may comprise an area for receiving the consumable, an aerosol generator, an aerosol generation area, a housing, a mouthpiece, a filter and/or an aerosol-modifying agent.

In some embodiments, the consumable for use with the non-combustible aerosol provision device may comprise aerosol-generating material, an aerosol-generating material storage area, an aerosol-generating material transfer component, an aerosol generator, an aerosol generation area, a housing, a wrapper, a filter, a mouthpiece, and/or an aerosol-modifying agent.

In some embodiments, the substance to be delivered may be an aerosol-generating material or a material that is not intended to be aerosolised. As appropriate, either material may comprise one or more active constituents, one or more flavours, one or more aerosol-former materials, and/or one or more other functional materials.

In some embodiments, the substance to be delivered comprises an active substance (sometimes referred to herein as an active compound).

The active substance as used herein may be a physiologically active material, which is a material intended to achieve or enhance a physiological response. The active substance may for example be selected from nutraceuticals, nootropics, psychoactives, or digiceutical or other technical/electronic devices that may induce a physiological response, such as vagus nerve stimulation (VGS). The active substance may be naturally occurring or synthetically obtained. The active substance may comprise for example nicotine, caffeine, taurine, theine, vitamins such as B6 or B12 or C, melatonin, cannabinoids, or constituents, derivatives, or combinations thereof. The active substance may comprise one or more constituents, derivatives or extracts of tobacco, cannabis or another botanical. In one embodiment, the active substance is a legally permissible recreational drug.

In some embodiments, the active substance comprises nicotine. In some embodiments, the active substance comprises caffeine, melatonin or vitamin B12.

As noted herein, the active substance may comprise one or more constituents, derivatives or extracts of cannabis, such as one or more cannabinoids or terpenes.

As noted herein, the active substance may comprise or be derived from one or more botanicals or constituents, derivatives or extracts thereof. As used herein, the term “botanical” includes any material derived from plants including, but not limited to, extracts, leaves, bark, fibres, stems, roots, seeds, flowers, fruits, pollen, husk, shells or the like. Alternatively, the material may comprise an active compound naturally existing in a botanical, obtained synthetically. The material may be in the form of liquid, gas, solid, powder, dust, crushed particles, granules, pellets, shreds, strips, sheets, or the like. Example botanicals are tobacco, eucalyptus, star anise, hemp, cocoa, cannabis, fennel, lemongrass, peppermint, spearmint, rooibos, chamomile, flax, ginger, ginkgo biloba, hazel, hibiscus, laurel, licorice (liquorice), matcha, mate, orange skin, papaya, rose, sage, tea such as green tea or black tea, thyme, clove, cinnamon, coffee, aniseed (anise), basil, bay leaves, cardamom, coriander, cumin, nutmeg, oregano, paprika, rosemary, saffron, lavender, lemon peel, mint, juniper, elderflower, vanilla, wintergreen, beefsteak plant, curcuma, turmeric, sandalwood, cilantro, bergamot, orange blossom, myrtle, cassis, valerian, pimento, mace, damien, marjoram, olive, lemon balm, lemon basil, chive, carvi, verbena, tarragon, geranium, mulberry, ginseng, theanine, theacrine, maca, ashwagandha, damiana, guarana, chlorophyll, baobab or any combination thereof. The mint may be chosen from the following mint varieties: Mentha Arventis, Mentha c.v., Mentha niliaca, Mentha piperita, Mentha piperita citrata c.v., Mentha piperita c. v, Mentha spicata crispa, Mentha cardifolia, Memtha longifolia, Mentha suaveolens variegata, Mentha pulegium, Mentha spicata c.v. and Mentha suaveolens

In some embodiments, the active substance comprises or is derived from one or more botanicals or constituents, derivatives or extracts thereof and the botanical is tobacco.

In some embodiments, the active substance comprises or derived from one or more botanicals or constituents, derivatives or extracts thereof and the botanical is selected from eucalyptus, star anise, cocoa and hemp.

In some embodiments, the active substance comprises or derived from one or more botanicals or constituents, derivatives or extracts thereof and the botanical is selected from rooibos and fennel.

Aerosolisable material, which also may be referred to herein as aerosol generating material, is material that is capable of generating aerosol, for example when heated, irradiated or energized in any other way. Aerosolisable material may, for example, be in the form of a solid, liquid or gel which may or may not contain nicotine and/or flavourants.

The aerosol-generating material may be an “amorphous solid”. In some embodiments, the amorphous solid is a “monolithic solid”. The aerosol-generating material may be non-fibrous or fibrous. In some embodiments, the aerosol-generating material may be a dried gel. The aerosol-generating material may be a solid material that may retain some fluid, such as liquid, within it. In some embodiments the retained fluid may be water (such as water absorbed from the surroundings of the aerosol-generating material) or the retained fluid may be solvent (such as when the aerosol-generating material is formed from a slurry). In some embodiments, the solvent may be water.

The aerosol-generating material may comprise one or more active substances and/or flavours, one or more aerosol-former materials, and optionally one or more other functional material.

The aerosol-former material may comprise one or more constituents capable of forming an aerosol. In some embodiments, the aerosol-former material may comprise one or more of glycerine, glycerol, propylene glycol, diethylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, and propylene carbonate.

The one or more other functional materials may comprise one or more of pH regulators, colouring agents, preservatives, binders, fillers, stabilizers, and/or antioxidants.

The material may be present on or in a support, to form a substrate. The support may, for example, be or comprise paper, card, paperboard, cardboard, reconstituted material, a plastics material, a ceramic material, a composite material, glass, a metal, or a metal alloy. In some embodiments, the support comprises a susceptor. In some embodiments, the susceptor is embedded within the material. In some alternative embodiments, the susceptor is on one or either side of the material.

A consumable is an article comprising or consisting of aerosol-generating material, part or all of which is intended to be consumed during use by a user. A consumable may comprise one or more other components, such as an aerosol-generating material storage area, an aerosol-generating material transfer component, an aerosol generation area, a housing, a wrapper, a mouthpiece, a filter and/or an aerosol-modifying agent. A consumable may also comprise an aerosol generator, such as a heater, that emits heat to cause the aerosol-generating material to generate aerosol in use. The heater may, for example, comprise combustible material, a material heatable by electrical conduction, or a susceptor.

An aerosol generator is an apparatus configured to cause aerosol to be generated from the aerosol-generating material. In some embodiments, the aerosol generator is a heater configured to subject the aerosol-generating material to heat energy, so as to release one or more volatiles from the aerosol-generating material to form an aerosol.

In some embodiments, the aerosol generator is configured to cause an aerosol to be generated from the aerosol-generating material without heating. For example, the aerosol generator may be configured to subject the aerosol-generating material to one or more of vibration, increased pressure, or electrostatic energy.

A mood state of a person (or user) may depend on a number of environmental factors such as work, exercise, gaming or any activity. Example embodiments described herein provide arrangements for determining current user mood states, predicting future mood states and influencing, managing or controlling mood states.

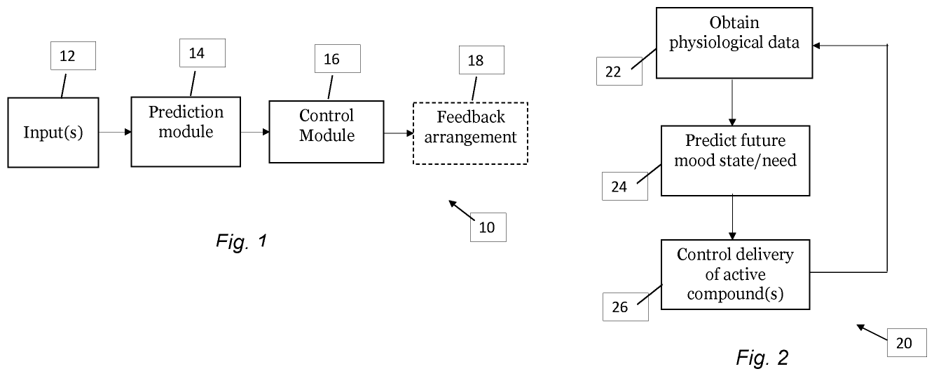

FIG. 1 is a block diagram of a system, indicated generally by the reference numeral 10, in accordance with an example embodiment. The system 10 comprises one or more inputs 12, a prediction module 14 and a control module 16. As discussed in detail below, the input(s) provide data (such as physiological data relating to a user) that enable a user mood state to be determined. The prediction module 14 enables a future mood state or need for the user to be determined or estimated. The control module 16 seeks to provide at least some control of the user mood state, for example by determining delivery of one or more active compounds (sometimes referred to herein as active substances) to influence, manage or control the mood state of the user.

The system 10 further comprises an optional feedback arrangement 18. The feedback arrangement 18 determines a response to delivered active compounds. The feedback arrangement may be implemented in a number of different ways. For example, the feedback arrangement may simply involve providing a number of iterations of a control algorithm, perhaps involving a wait state to allow for the impact of a delivery active compound to be apparent.

FIG. 2 is a flow chart showing an algorithm, indicated generally by the reference numeral 20, in accordance with an example embodiment. The algorithm 20 may be implemented using the system 10 described above.

The algorithm 20 starts at step 22, where physiological data is obtained. At least some of the physiological data may suitable for use in determining a mood state of a user. The physiological data may be (or may be derived from) the input(s) 12 described above. As discussed further below, at least some of those inputs may be obtained from one or more sensors, thereby providing real-time physiological data (such as one or more of: ECG or EKG data, EEG data, temperature, oxygen usage, and eye movement data).

At operation 24, a future mood state or need of a user is determined based, at least in part, on the (real-time) physiological data obtained in the operation 22. The future mood state may be generated by the prediction module 14 of the system 10. As discussed further below, the prediction module 14 may comprise an artificial intelligence or machine learning model such as a trained neural network. That neural network may be configured to recognise and predict user mood states.

At operation 26, delivery of one or more active compounds (to the user) is controlled. The active compounds may be controlled to influence, manage or control the mood state of the user. The operation 26 may be implemented using the control module 16 of the system 10.

The operation 26 may include determining/selecting one or more active compounds to be delivered to the user. The operation 26 may include determining timing of delivery of selected active compound(s) (e.g. a delivery start time and a delivery duration). The operation 26 may include determining a delivery dose of selected active compound(s). Selection of active compound(s) may be based on a-priori knowledge of their effects on user mood. Thus, the delivery of the selected compound(s) can be defined to seek to address a need predicted in the operation 24. Some example delivery mechanisms are discussed below.

There are many active compounds, or combinations of active compounds, that may be delivered under the control of the algorithm 20. Examples include melatonin (e.g. to aid sleep), caffeine (e.g. to aid focus or alertness or to provide energy) and/or cannabidiol (CBD) (e.g. to aid relaxation). The skilled person will be aware of many other compounds (or combinations of compounds) that could be used (including other active compounds mentioned herein).

With the delivery of active compounds controlled, the algorithm 20 returns to operation 22, where further physiological data is obtained. Thus, the algorithm 20 is iterative, such that a user response to the delivery of delivered active compounds can be monitored and used to update the delivery of active compounds in the future. It should be noted that a delay or “wait state” may be required in order to wait for an impact of a delivered compound to become apparent.

The iterative nature of the algorithm 20 may provide the feedback arrangement 18 of the system 10 described above. The control module 16 may, for example, implement a step of determining a response to delivered active compounds. That step may be a separate step (not shown in the algorithm 20), but could be implemented as part of the prediction of the future mood state/need (step 24) or the control of delivery of active compound(s) (step 26).

The system 10 and/or the algorithm 20 may be used to influence, manage or control a mood state of a user who is within a virtual world. In some circumstances, controlling the mood state of a user in a virtual world may enable an immersive experience of the user to be enhanced.

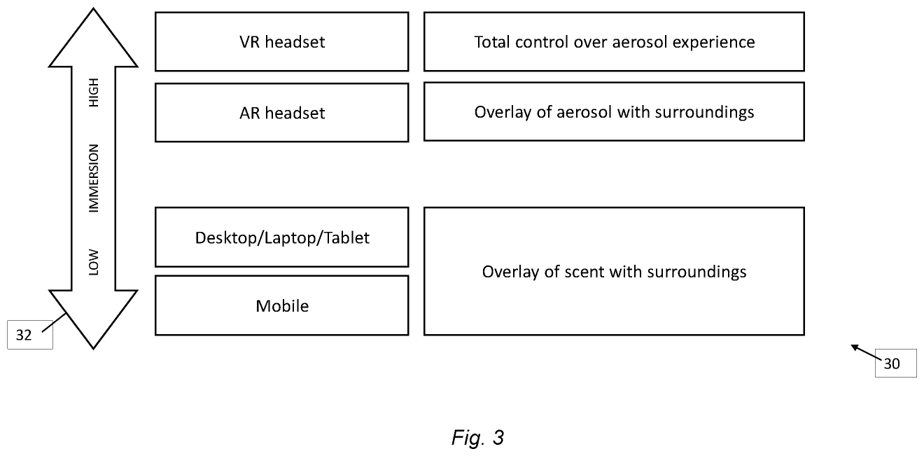

FIG. 3 is a block diagram, indicated generally by the reference numeral 30, demonstrating example types of immersion in accordance with an example embodiment.

The block diagram 30 includes a scale 32 indicating an immersion level of a user. The highest level of immersion may occur with the user wearing a virtual reality (VR) headset. The use of virtual reality enables video content to be provided to a user using VR display system. The displayed content represents a VR space or world for immersive output through the display system. In some embodiments, audio is provided and a VR headset may be configured to provide VR video and audio content to the user, e.g. through the use of a pair of video screens and headphones incorporated within the headset.

Augmented reality (AR) refers to a real-world view that is augmented by computer-generated sensory input. Since the real-world view remains visible, the degree of immersion provided by AR is generally less than VR (as indicated by FIG. 3).

The provision of displays using a monitor of a computer (desktop, laptop or tablet) or a mobile device (e.g. a smartphone) offers a lower degree of immersion. Nevertheless, the control of mood can increase an overall immersive experience in such circumstances.

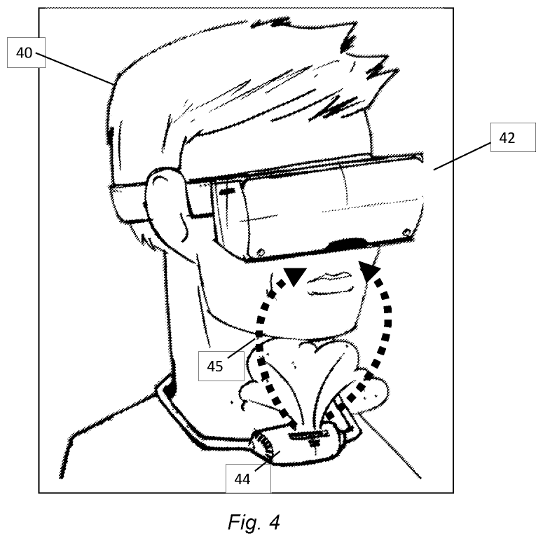

FIG. 4 shows a user 40 wearing a headset 42 (e.g. a VR, AR or XR headset) and inhaler 44 in accordance with an example embodiment. The inhaler 44 takes the form of a neck-wearable aerosol generating device. The headset 42 and inhaler 44 allow the user 40 to connect to a virtual world (or metaverse). The headset 42 provides visual and audio stimuli to the user 40 whilst the inhaler 44 provides an aerosol (e.g. delivering one or more selected active compounds).

In some embodiments, the headset 42 comprises sensors (not shown) which collect physiological data relating to the user. In some embodiments, the headset 42 implements the prediction module 14 and the control module 16 described above (although those functions could also be provided either totally or partially outside the headset). The control module may be configured to select one or more active compound(s) from an active selection module (not shown). The selected compound(s) may be provided to a heater (not shown) to generate an aerosol that is released towards the nose of the user as indicated by arrows 45. In some embodiments, the inhaler 44 releases the active compound(s)/aerosol synchronously with visual and/or audio stimuli provided by the VR headset 42. In some embodiments, the inhaler 44 releases the active compound(s)/aerosol synchronously with the data collected by the sensor(s). In some embodiments, the sensor(s), prediction module, controller, active selection module and/or heater of the system 10 may be part of either the VR headset 42 or the inhaler 44 or part of one or more separate devices or a combination thereof. In some embodiments, the inhaler 44 is a mouth wearable device. In some embodiments, the inhaler 44 is part of the VR headset 42, an AR headset or any other headset.

In some example embodiments, aerosol delivery may be linked or co-ordinated with visual and/or audio content of the virtual world to provide a high level of immersion for the user 40.

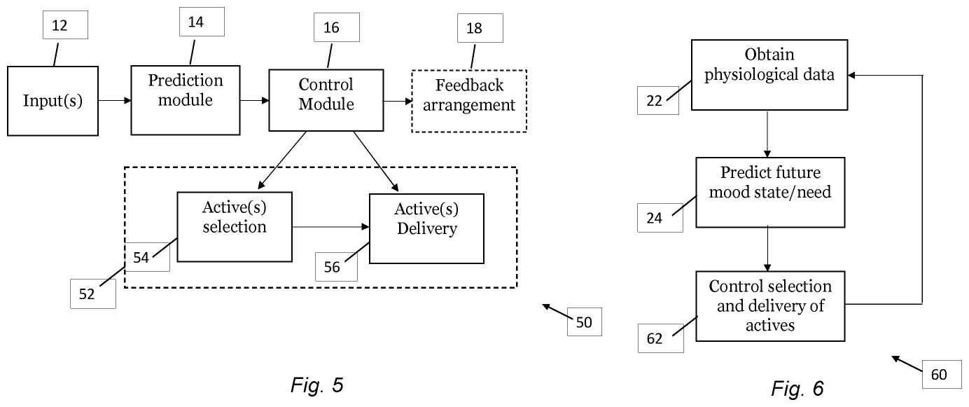

FIG. 5 is a block diagram of a system, indicated generally by the reference numeral 50, in accordance with an example embodiment.

The system 50 comprises the one or more inputs 12, the prediction module 14 and the control module 16 of the system 10 described above. The one or more inputs may be provided by one or more sensors providing real-time physiological data.

The system 50 further comprises a delivery mechanism 52 for implementing delivery of one or more active compounds, as determined/selected by the control module 16. The example delivery mechanism 52 comprises an active(s) selection module 54 and an active(s) delivery module 56. As discussed above, the prediction module 14 enables a future mood state or need of the user to be determined or estimated and the control module 16 seeks to provide at least some control of the user mood state. The system 50 further comprises the optional feedback arrangement 18.

FIG. 6 is a flow chart showing an algorithm, indicated generally by the reference numeral 60, in accordance with an example embodiment. The algorithm 60 may be implemented using the system 50 described above. The algorithm 60 includes many of the features of the algorithm 20 described above.

The algorithm 60 starts at step 22, where, as discussed above, physiological data is obtained. The physiological data may be (or may be derived from) the input(s) 12 described above (for example, from one or more sensors), thereby providing real-time physiological data, for example to the prediction module 14.

At operation 24, as discussed above, a future mood state or need of a user is determined based, at least in part, on the (real-time) physiological data obtained in the operation 22.

At operation 62, the selection and delivery of one or more active compounds (to the user) is controlled. The operation 62 thereby implements the operation 26 of the algorithm 20 described above

In the operation 62, the control module 16 controls the selection and delivery of one or more active compounds by the delivery mechanism 52 to the user. As with the operation 26 described above, the operation 66 may include determining and/or selecting one or more active compounds (such as one or more of the active compounds discussed above) to be delivered to the user and may include determine timing of delivery of selected active compounds (e.g. a delivery state time and a delivery duration). The control module 16 may comprise a look up table providing the intended effect of each active compound available to the system such as stress relief etc. The look up table may further comprise details of the advised delivery time and delivery duration of each active compound in order to achieve the intended effect. The look up table may therefore be used in the selection of active compounds to meet a need and/or in determining the delivery parameters of a selected active compound to meet a need.

With the delivery of active compounds controlled, the algorithm 60 returns to the operation 62, thereby providing a feedback loop.

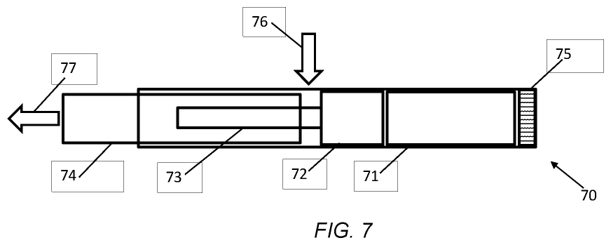

FIG. 7 is a block diagram of an aerosol generating device, indicated generally by the reference numeral 70, in accordance with an example embodiment. The aerosol generating device 70 may be used as, or form part of, the active(s) delivery module 56 described above.

The aerosol generating device 70 comprises a battery 71, a control circuit 72, a heater 73 and a consumable 74. The device also includes a connector 75 (such as a USB connector). The connector 75 may enable connection to be made to a power source for charging the battery 71, for example under the control of the control circuit 72. The control circuit 72 may form part of (or being under the control of) the control module 16 described above.

In the use of the device 70, the heater 73 is inserted into the consumable 74, such that the consumable may be heated to generate an aerosol. In the use of the device 70, air is drawn into the device 70, through an air inlet as indicated by arrow 76, then passes through the consumable, delivering the aerosol to the user as indicated by the arrow 77. The aerosol generating device 70 is provided by way of example only. Many alternative aerosol generating devices may be used in example implementations of the principles described here. For example, the aerosol generating device 70 may have access to multiple active compounds and include a mechanism for selecting between active compounds for delivery, as discussed further below. Such multiple active compounds could be mixed into bespoke formulations, for example based on past used experience. Furthermore, the aerosol generating device 70 may be replaced with an alternative device for delivering active compounds in the form of a mist or spray. Other suitable arrangements will be apparent to those of ordinary skill in the art.

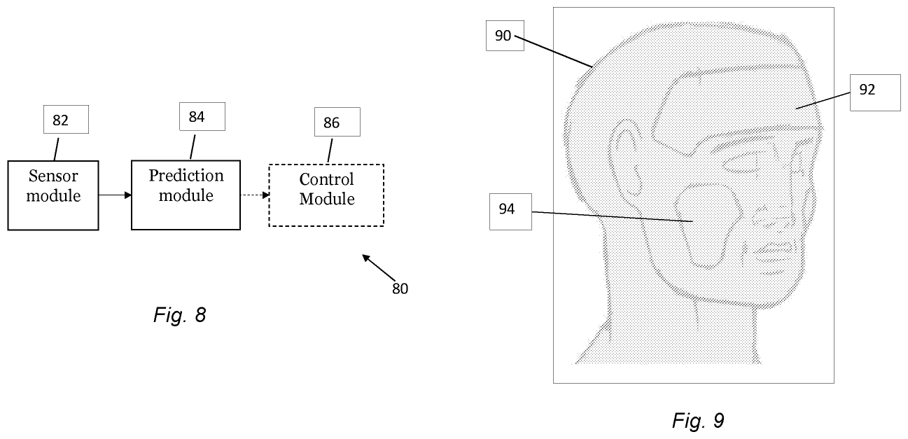

FIG. 8 is a block diagram of a system, indicated generally by the reference numeral 80, in accordance with an example embodiment. The system 80 comprises a sensor module 82 and a prediction module 84. The system 80 may further comprise an optional control module 86.

The sensor module 82 comprises one or more sensors for obtaining real-time physiological data suitable for use in determining a mood state of a user. The sensor module 82 may therefore provide the one or more inputs 12 described above.

The prediction module 84 receives and processes the output of the sensor module 82. The prediction module 84 is an example implementation of the prediction module 14 described above. Similarly, the optional control module 86 is an example implementation of the control module 16 described above.

FIG. 9 shows a user 90 in accordance with an example embodiment. The user 90 is shown with a first data collection point 92 and a second data collection point 94 on the user's face.

FIG. 9 illustrates the parts of a human face that may be used as data collection points for one or more sensors (e.g. one or more sensors of the sensor module 82) to provide real-time physiological data for determining a mood state of the user. The sites may include the skin areas of the forehead 92 or the cheeks 94 of the face. Example sensors include thermal sensors, non-thermal sensors, galvanic skin response (GSR) sensor, eye tracking/dilation sensors, transdermal optical imaging (TOI) sensors, and other sensors to detect brain activity etc. As discussed further below, one more sensors may be integrated into, or otherwise form part of, a headset (e.g. a virtual reality, augmented reality or extended reality headset).

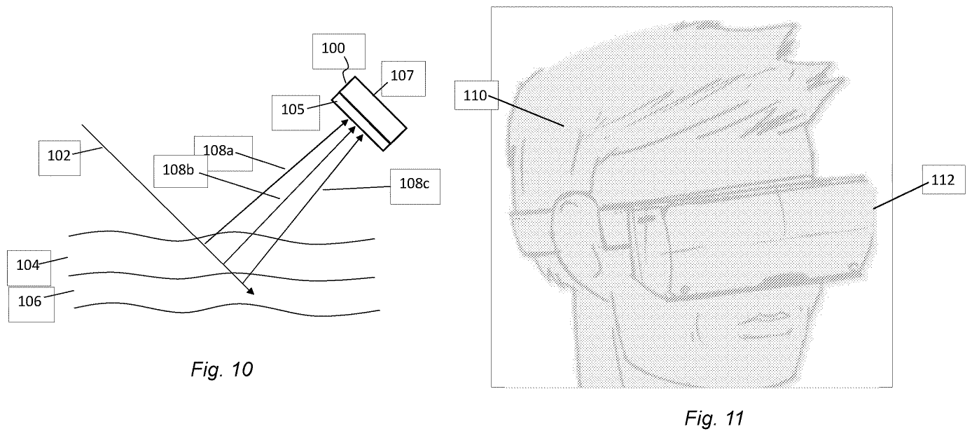

FIG. 10 shows a transdermal optical imaging (TOI) sensor 110 used in some example embodiments. By way of example, the sensor module 82 may comprise one or more TOI sensors 110.

The sensor 110 arranged to capture light reflected off/re-emitted by human skin. The skin may be parts of a human face such as skin areas 92 and 94 described above with reference to FIG. 9. In some embodiments, the sensor 110 is an optical camera configured for Transdermal Optical Imaging (TOI).

In TOI, light 102 is directed towards the skin. Part of the light 102 is absorbed in a melanin layer of the skin 104. Part of the light 102 is absorbed in a haemoglobin layer of the skin 106. In the example embodiment shown in FIG. 10, light of specific wavelengths (e.g. blue light) 108a is re-emitted by the melanin layer 104, light of specific wavelengths (e.g. green light) 108b is re-emitted by the melanin layer 104 and light of specific wavelengths (e.g. red light) 108c is re-emitted by the haemoglobin layer 106. The optical camera 100 may comprise an aperture 105 mounted onto a headset (such as the headset 112 described below) and arranged to capture the re-emitted blue light 108a, green light 108b and red light 108c. In some embodiments, the optical camera 100 comprises a filter (e.g. a Bayer filter) with an array of red, green and blue photosensors (not shown). In some embodiments, the optical camera 100 further comprises a processor 107 configured to process the red light captured by the red photosensors. The processor 107 may be configured to perform TOI processing to provide heart rate and heart rate variability analytics of the user. In some embodiments, the heart rate and heart rate variability analytics are provided to a prediction module (such as prediction modules 14 or 84 described above) to predict a future mood state of the user.

FIG. 11 shows a user 110 wearing a headset 112 in accordance with an example embodiment. The headset 112 may be a VR, AR, mixed reality or extended reality headset which may be the same or similar to the headset 42 described above with reference to FIG. 4. The headset 112 may be configured to immerse the user 110 in a virtual world such as a metaverse. The virtual world/metaverse may be configured to affect the mood state of the user. The headset 112 comprises one or more sensors (not shown) which are arranged to measure real-time physiological data of the user 110. The data collected is provided to a prediction module (such as the prediction modules 14 and 84 described above). The headset 112 may therefore provide some or all of the sensors modules 82, as noted above.

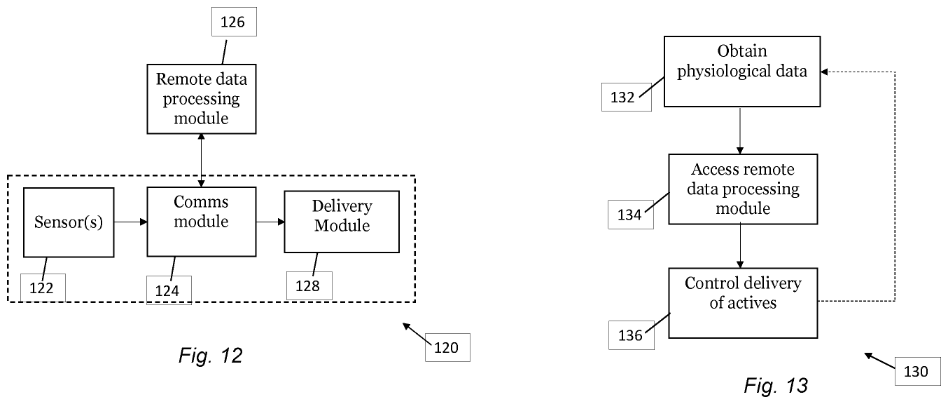

FIG. 12 is a block diagram of a system, indicated generally by the reference numeral 120, in accordance with an example embodiment.

The system 120 comprises one or more sensors 122, a communication module 124, a remote data processing module 126 and a delivery mechanism 128. The one or more sensors may take many forms, such as transparent optical imaging cameras, thermal sensors and non-thermal sensors, as discussed above. At least some of said sensors may be mounted within a user headset (e.g. a virtual reality, augmented reality, mixed reality or extended reality headset). The sensor(s) 122, communication module 124 and delivery mechanism 128 may be provided at or near a user (e.g. at or near a headset being worn by a user); the remote data processing module 126 may be provided elsewhere and may be accessed, for example, over a network (such as the Internet).

FIG. 13 is a flow chart showing an algorithm, indicated generally by the reference numeral 130, in accordance with an example embodiment. The algorithm 130 may be implemented using the system 120 described above.

The algorithm 130 starts at operation 132, where real-time physiological data is obtained (e.g. by the one or more sensors 122). The real-time physiological data is for use in determining a mood state of a user, as discussed above. In some example embodiments, the user is in a virtual world (and may, for example, be wearing a user headset, as discussed above).

At operation 134, a remote data processing module (e.g. the remote data processing module 126) is accessed to obtain input in response to the sensor data obtained in the operation 132. The remote data processing module comprises a model (not shown in FIG. 12) for determining a future mood state of the user based on said sensor data. The model may, for example, comprises an artificial intelligence or machine learning model. Providing remote access to the model may reduce local storage requirements and may make it easier to update the model. Note that the model may be made available to multiple users.

At operation 136, the delivery of one or more active compounds is controlled (e.g. by the delivery mechanism 128) based on the input received from said remote data processing module. The one or more active compounds are delivered to influence, manage or control the mood state of said user.

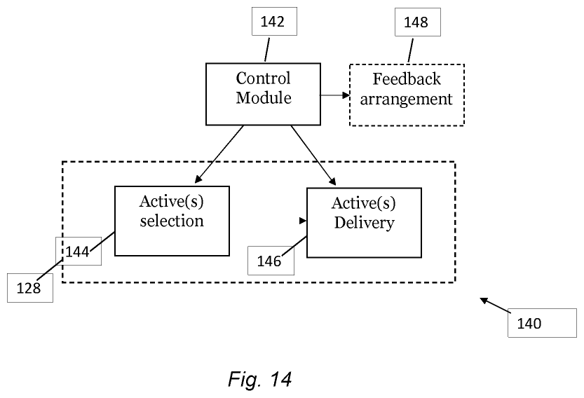

FIG. 14 is a block diagram of a system, indicated generally by the reference numeral 140, in accordance with an example embodiment.

The system 140 includes the delivery mechanism 128 of the system 140 and further comprises a control module 142 and a feedback arrangement 148. In the example system 140, the delivery mechanism 128 comprises an active compound(s) selection module 144 and an active compound(s) delivery module 146. The delivery module 146 may comprise an aerosol delivery mechanism, as discussed above.

In the system 140, the control module 142 controls the delivery mechanism 128. For example, in the system 140, the control module 142 may control the selection of one or more active components for delivery (by controlling the active compound(s) selection module 144) and may also control the delivery of the selected compound(s) (by controlling the active compound(s) delivery module 146).

The control module 412 may control active compound delivery based on the input received at the communication module 124. Alternatively, the control module 142 may form part of the remote data processing module 126.

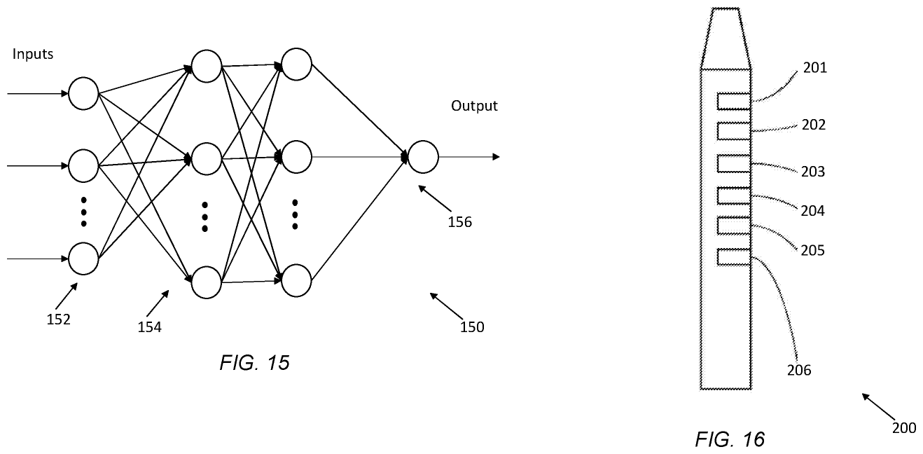

FIG. 15 is a block diagram of a neural network, indicated generally by the reference numeral 150, used in some example embodiments. For example, one or more of the prediction modules described above may be implemented using the neural network 150. The neural network 150 comprises an input layer 152, one or more hidden layers 154, and an output layer 156. At the input layer 152, data (such as sensor data) is received as an input. The hidden layers 154 may comprise a plurality of hidden nodes, where received sensor data are processed. At the output layer 156, one or more outputs (such as a predicted future state or need) are output.

The inputs to the model may the outputs of the one or more sensors described above. The output of the model 150 may be a future mood state or need for a user.

The model 150 may be trained based on training data of known or simulated sensor data and need states. The training may comprise re-enforcement learning or some similar technique.

The sensor(s) described above provide real-time data (such as physiological data relating to a user) to a control module (such as the control modules 16, 86 or 142). The sensors may take many forms. For example, the sensors (either the remote sensors or the headset sensors) may comprise one or more imaging devices or cameras. Alternatively, or in addition, the sensors (either the remote sensors or the headset sensors) may comprise Internet of Things (IOT) devices. The sensors may form part of the delivery mechanism or part of an accessory to said delivery mechanism. Indeed, the sensors may include any sensor that transduces a physiological trait or characteristic into an electrical signal that can be processed to determine, measure or track that particular trait or characteristic. This includes sensors that are cameras, microphones, electrophysiological sensors (EEG, ECG etc.), temperature sensors etc. Sensors can also be used to detect behavioural signals such as handwriting, voice and facial characteristics. Positioning data (such as GPS data or a location within a virtual space) could be used to assess a mood state.

As noted above, the aerosol generating device 70 is provided by way of example only. Many alternative aerosol generating devices may be used in example implementations of the principles described here. For example, the aerosol generating device 70 may have access to multiple active compounds and include a mechanism for selecting between active compounds for delivery.

FIG. 16 is a block diagram showing an aerosol delivery mechanism, indicated generally by the reference numeral 200, in accordance with an example embodiment.

In broad outline, the device 200 is configured to generate an aerosol for delivery to a user from at least one aerosolisable material received within the device 200. Herein an aerosolisable material includes any material that may be aerosolised. In the examples discussed herein the aerosol provision device 200 is configured to receive a plurality of aerosolisable materials, where each aerosolisable material is housed in or forms a consumable, e.g., the consumable may be a container housing the aerosolisable material. The aerosol provision device 200 is configured to receive at least a first consumable 201 and a second consumable 202, and the device 200 may also be configured to receive further consumables 203, 204, 205 and 206. Herein, reference is made to the device 200 receiving consumables 201 to 206; however, it should be appreciated that device 200 more generally receives a plurality of aerosolisable materials. In some implementations, the aerosolisable materials may be provided detached from one another (e.g., as separate consumables as described herein) or may be provided on a common substrate as a single consumable to be received in the device 200.

The aerosol delivery mechanism 200 may be configured to recognise the identity and position of consumables 201 to 206 received in the device 200 and may transmit data indicating the identity and position of consumables received in the device 200 (e.g. to a control module, such as the control module 16, 86 or 142 described above). The consumables 201 to 206 may, for example, comprise radio frequency identification (RFID) tags that may be used for identification purposes.

The aerosol delivery mechanism 200 allows for a usage session which is appropriate for the consumables received within the device 200 to be implemented. Appropriate settings may be applied to the aerosol delivery mechanism 200 depending on the consumables inserted and depending on the contextual environment of the user (e.g. depending on a determined or predicted user need). Herein, reference is made to example devices transmitting data regarding the identity and position of consumables or, more generally, aerosolisable materials received in the device. It should be appreciated that in some implementations, a device may recognise the identity and/or position of consumables/materials received in the device and a controller or the like in the device may use the identity and position data to provide instructions to the device for producing an aerosol based on the identity and/or position of consumables/materials received in the device.

The device 200 comprises means for receiving at least the first consumable 201 for containing a first aerosolisable material, and for receiving the second consumable 202 for containing a second material. In some examples, the device is configured to receive further consumables, such as third 203, fourth 204, fifth 205, and sixth 206 consumables for containing third, fourth, fifth and sixth aerosolisable materials respectively. In other examples, the device 200 may be configured to receive any number, two or more, of consumables.

Aerosol is generated by the device 200 from at least the first consumable 201 containing first aerosolisable material. The first consumable 201 is in fluidic contact with a central aperture (for example via a value or flow device, not shown), and air flowing in through one or more air inlets mixes with aerosol generated from the first consumable 201 to generate a flow of aerosol. The aerosol flow is drawn towards the outlet for delivery to the user. In some examples, air flowing from the air inlets to the mouthpiece may pass through each consumable or aerosolisable material received in the device sequentially. That is, each of the consumables or aerosolisable materials in the device may be located on the same air flow path between the air inlets and the mouthpiece. In other examples, there may be multiple branches for air flowing from the air inlet/s towards the outlet. For example, a plurality of branches may be provided and each branch of the plurality of branches may pass through one or more of the consumables or aerosolisable materials. There may be one branch for each of the consumables or aerosolisable materials, or each air flow path may pass through more than one of the consumables or aerosolisable materials. In some examples, where there are multiple air flow branches there may be a branch which does not pass through a consumable or aerosolisable material. Where there are multiple air flow branches the branches may join, in an admixing chamber or the like, prior to aerosol flowing to the mouthpiece.

The second consumable 202 may also produce aerosol which mixes with the aerosol generated from the first consumable 201 before the aerosol reaches the outlet. For example, the second consumable 202 may produce a flavoured aerosol. Additionally or alternatively, one or more properties of the aerosol generated from the first consumable 201 may be modified by material contained by the second consumable 202 and, optionally, by material contained by one or more further consumables 203, 204, 205, etc. received within the device.

In some example embodiments, the aerosolisable materials may be liquids or gels; however this is not essential to all example embodiments.

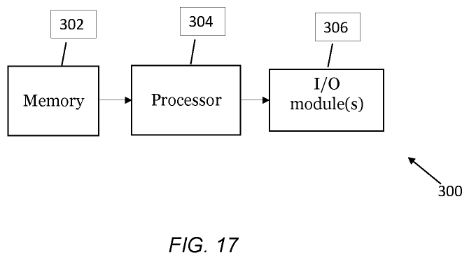

FIG. 17 is a block diagram of a processing system, indicated generally by the reference numeral 300, that may be used to implement one or more of the example embodiments described previously. The processing system 300 may, for example, be (or may include) the apparatus referred to in the claims below.

The processing system 300 may have a processor 304, a memory 302 coupled to the processor (e.g. comprising a random access memory (RAM) and/or a read only memory (ROM)). The processing system 300 may also comprise one or more input/output (I/O) modules 306, such as one or more user interface modules.

The memory 302 may comprise code which, when executed by the processor 304 implements aspects of the methods and algorithms described herein.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention. Various embodiments of the invention may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in future.

1. A system comprising:
one or more sensors for obtaining real-time physiological data for use in determining a mood state of a user;
communicating means for providing said sensor data to a remote data processing module and for receiving input from said remote data processing module, wherein the remote data processing module comprises a model for determining a future mood state of the user based on said sensor data; and
a delivery mechanism for delivering one or more active compounds to influence, manage or control the mood state of said user, wherein delivery of said compounds is controlled based on the input received from said remote data processing module.

2. A system as claimed in claim 1, further comprising a control module for determining said active compound delivery based, at least in part, on the input received from said remote data processing module.

3. A system as claimed in claim 1, wherein said remote data processing module comprises a control module for determining said active compound delivery of said active compounds.

4. A system as claimed in claim 1, wherein, in use, said user is in a virtual world.

5. A system as claimed in claim 1, wherein said sensors are mounted within a user headset.

6. A system as claimed in claim 5, wherein said headset is a virtual reality, augmented reality, mixed reality or extended reality headset.

7. A system as claimed in claim 1, wherein said sensors comprise one or more of:
one or more transparent optical imaging cameras; one or more thermal sensors; and
one or more non-thermal sensors.

8. A system as claimed in claim 1, wherein the remote data processing module comprises an artificial intelligence or machine learning model.

9. A system as claimed in claim 1, wherein said delivery module comprises an aerosol delivery mechanism.

10. A method comprising:
obtaining real-time physiological data for use in determining a mood state of a user;
providing said sensor data to a remote data processing module, wherein the remote data processing module comprises a model for determining a future mood state of the user based on said sensor data;
receiving input from said remote data processing module; and
delivering one or more active compounds to influence, manage or control the mood state of said user, wherein delivery of said compounds is controlled based on the input received from said remote data processing module.

11. A method as claimed in claim 10, further comprising determining said active compound delivery based, at least in part, on the input received from said remote data processing module.

12. A method as claimed in claim 10, wherein, in use, said user is in a virtual world.

13. A method as claimed in claim 10, wherein the remote data processing module comprises an artificial intelligence or machine learning model.

14. A method as claimed in claim 10, wherein said delivery module comprises an aerosol delivery mechanism.

15. A computer program comprising instructions for causing an apparatus to perform at least the following:
obtaining real-time physiological data for use in determining a mood state of a user;
providing said sensor data to a remote data processing module, wherein the remote data processing module comprises a model for determining a future mood state of the user based on said sensor data;
receiving input from said remote data processing module; and
delivering one or more active compounds to influence, manage or control the mood state of said user, wherein delivery of said compounds is controlled based on the input received from said remote data processing module.
